(19) United States
(12) Patent Application Publication
(10) Pub. No.: US 2023/0031403 A1
(43) Pub. Date: Feb. 2, 2023

(54) ROBOTIC SURGICAL ASSEMBLIES

(71) Applicant: Covidien LP, Mansfield, MA (US)

(72) Inventors: Michael A. Zemlok, Prospect, CT (US); Ranjan Kumar Mishra, Orange, CT (US); Mark H. MacLeod, Southbury, CT (US); Jaimeen V. Kapadia, Cambridge, MA (US)

(21) Appl. No.: 17/789,850

(22) PCT Filed: Jan. 19, 2021
(86) PCT No.: PCT/US2021/013852

§ 371 Date: Jun. 29, 2022

Related U.S. Application Data
(60) Provisional application No. 62/970,267, filed on Feb. 5, 2020.

(51) Int. Cl. A61B 34/30; A61B 34/00

(52) U.S. Cl. CPC A61B 34/30; A61B 34/71; A61B 2017/00477

(57) ABSTRACT

A robotic surgical system includes an instrument drive unit, a surgical instrument drivingly coupled to the instrument drive unit, and a carriage for supporting the instrument drive unit and the attached surgical instrument. The carriage includes a drive assembly that drives a rotation of the surgical instrument about a longitudinal axis defined by the surgical instrument. The carriage further includes a locking assembly configured to selectively fix the surgical instrument in a rotational orientation.

### FIELD

The present technology is generally related to robotic surgical systems used in minimally invasive medical procedures.

### BACKGROUND

Robotic surgical systems have been used in minimally invasive medical procedures. Some robotic surgical systems include a console supporting a surgical robotic arm and a surgical instrument, having at least one end effector (e.g., forceps or a grasping tool), mounted to the robotic arm. The robotic arm provides mechanical power to the surgical instrument for its operation and movement. To use each unique surgical instrument with the robotic surgical system, an instrument drive unit is provided to interface with the selected surgical instrument to drive operations of the surgical instrument. The surgical instrument may be attached to the instrument drive unit in a way that allows for rotation of the surgical instrument about its longitudinal axis.

### SUMMARY

In accordance with an aspect of the present disclosure, a robotic surgical assembly is provided. The robotic surgical assembly includes an instrument drive unit configured to drive operations of a surgical instrument and a carriage configured to be slidably coupled to a robotic arm and to support the instrument drive unit. The carriage includes a drive assembly and a locking assembly. The drive assembly has a motor, a pulley operably coupled to the motor, and a drive belt coupled to the pulley and configured to be operably coupled to the surgical instrument, such that the surgical instrument rotates in response to a rotation of the pulley. The locking assembly is configured to selectively lock the drive assembly to prevent rotation of the surgical instrument.

In aspects, the locking assembly may include a lock pad disposed adjacent a first side of the drive belt, and a lever arm having a first end disposed on a second side of the drive belt. The lever arm may be configured to pivot between a first position, in which the drive belt is in spaced relation to the lock pad, and a second position, in which the first end of the lever arm presses the drive belt into engagement with the lock pad to resist travel of the drive belt.

In aspects, the first side of the drive belt may have a plurality of teeth, and the lock pad may have a tooth configured for receipt in adjacent teeth of the drive belt when the lever arm is in the second position.

In aspects, the carriage may include a housing defining a slot having a first end and a second end. The first end of the slot may be configured for detachable receipt of a second end of the lever arm when the lever arm is in the first position. The second end of the slot may be configured for detachable receipt of the second end of the lever arm when the lever arm is in the second position.

In aspects, the instrument drive unit may include a housing configured to be rotationally fixed relative to the carriage and a motor pack rotatably supported in the housing.

In aspects, the motor pack may be configured to non-rotatably couple to the surgical instrument.

In aspects, the drive belt may be configured to be non-rotatably coupled to the motor pack, such that the motor pack and the surgical instrument rotate about a longitudinal axis defined by the surgical instrument upon rotation of the pulley.

In aspects, the locking assembly may include a solenoid and a pawl associated with the solenoid and disposed adjacent the pulley. The solenoid may be configured to move the pawl between a first position, in which the pawl is disengaged from the pulley, and a second position, in which the pawl is engaged to the pulley to resist rotation of the pulley.

In aspects, the solenoid may include a housing and a plunger coupled to the housing. The plunger may have an end fixed to the pawl, such that the pawl moves between the first and second positions in response to movement of the plunger relative to the housing.

In aspects, the pawl may have an arcuate surface defining a tooth configured to engage the pulley when the pawl is in the second position.

In accordance with another aspect of the disclosure, a robotic surgical system is provided. The robotic surgical system includes a surgical instrument defining a longitudinal axis, an instrument drive unit configured to transmit rotational forces to the surgical instrument while the surgical instrument is coupled to the instrument drive unit, and a carriage supporting the instrument drive unit and the surgical instrument while the surgical instrument is coupled to the instrument drive unit. The instrument drive unit has a motor pack configured to non-rotatably couple to the surgical instrument. The carriage includes a back member, a housing extending from the back member, a drive assembly, and a locking assembly. The drive assembly has a motor supported in the back member, a pulley supported in the housing and operably coupled to the motor, and a drive belt supported in the housing and coupled to the pulley. The drive belt is configured to non-rotatably couple to the motor pack, such that the surgical instrument rotates in response to a rotation of the pulley. The locking assembly is configured to selectively lock the drive assembly to prevent rotation of the surgical instrument.

In aspects, the housing of the carriage may define a slot having a first end configured for detachable receipt of a second end of the lever arm when the lever arm is in the first position, and a second end configured for detachable receipt of the second end of the lever arm when the lever arm is in the second position.

In aspects, the robotic surgical system may further include a rail configured to slidably support the back member of the carriage thereon.

In aspects, the locking assembly may include a slider slidably supported in the housing and defining a cam slot, and a brake having a projection received in the cam slot. The slider may be configured to move the brake between a first position, in which the motor pack is free to rotate, and a second position, in which the brake resists rotation of the motor pack.

In aspects, the brake may move between the first and second positions along an axis that is perpendicular to a longitudinal axis defined by the slider.

Other aspects, features, and advantages will be apparent from the description, the drawings, and the claims that follow.

### BRIEF DESCRIPTION OF THE DRAWINGS

Aspects of the present disclosure are described herein with reference to the accompanying drawings, wherein:

FIG. 1 is a schematic illustration of a robotic surgical system including a robotic surgical assembly in accordance with the present disclosure;

FIG. 2 is a front, perspective view of the robotic surgical assembly of FIG. 1 shown supported on a slide rail of the robotic surgical system;

FIG. 3 is a cross-section, taken along lines 3-3 in FIG. 2, illustrating a motor assembly of an instrument drive unit of the robotic surgical assembly;

FIG. 4A is a perspective view, with parts separated, of a carriage of the robotic surgical assembly of FIG. 2;

FIG. 4B is a perspective view of the carriage of FIG. 4A;

FIG. 5 is a cross-section, taken along lines 5-5 in FIG. 4B, illustrating a drive assembly of the carriage for driving a rotation of the motor assembly of FIG. 3;

FIG. 6 is a perspective view, with parts separated, of the drive assembly of FIG. 5;

FIG. 7 is a bottom, perspective view, with parts removed illustrating the drive assembly of FIG. 5 and an associated locking assembly of the carriage;

FIG. 8 is a bottom view illustrating the drive assembly of FIG. 7;

FIG. 9 is a bottom, perspective view, with parts shown in phantom, of the drive assembly and locking assembly of FIG. 7;

FIG. 10A is a bottom view illustrating another embodiment of a locking assembly shown in a non-locking state with the drive assembly of FIG. 5;

FIG. 10B is a bottom view illustrating the locking assembly of FIG. 10A shown in a locking state with the drive assembly of FIG. 5;

FIG. 11A is a bottom view illustrating yet another embodiment of a locking assembly shown in a non-locking state with the drive assembly of FIG. 5;

FIG. 11B is a bottom view illustrating the locking assembly of FIG. 11A shown in a locking state with the drive assembly of FIG. 5;

FIG. 12 is an enlarged, bottom view, with parts shown in phantom and parts removed, of yet another embodiment of a locking assembly of the carriage;

FIG. 13 is a bottom view, with parts removed, of the locking assembly of FIG. 12;

FIG. 14 is a perspective view illustrating a pivotable brake of the locking assembly of FIG. 12;

FIG. 15A is a bottom view illustrating yet another embodiment of a locking assembly shown in a non-locking state with the drive assembly of FIG. 5;

FIG. 15B is a bottom view illustrating the locking assembly of FIG. 15A shown in a locking state with the drive assembly;

FIG. 16A is a bottom view illustrating yet another embodiment of a locking assembly shown in a non-locking state with the drive assembly of FIG. 5;

FIG. 16B is a bottom view illustrating the locking assembly of FIG. 16A shown in a locking state with the drive assembly;

FIG. 17 is a side, perspective view of a carriage illustrating another embodiment of a locking mechanism of the carriage;

FIG. 18 is a longitudinal cross-sectional view of the carriage of FIG. 17;

FIG. 19 is a side, perspective view of a carriage illustrating yet another embodiment of a locking mechanism; and

FIG. 20 is a longitudinal cross-sectional view of the carriage of FIG. 19.

### DETAILED DESCRIPTION

Aspects of the presently disclosed robotic surgical assembly including a carriage and an instrument drive unit for driving the operation of an electromechanical surgical instrument and methods thereof are described in detail with reference to the drawings, in which like reference numerals designate identical or corresponding elements in each of the several views.

As used herein, the term “distal” refers to that portion of the robotic surgical system, or component thereof, that is closer to a patient, while the term “proximal” refers to that portion of the robotic surgical system, or component thereof, that is further from the patient.

As used herein, the terms parallel and perpendicular are understood to include relative configurations that are substantially parallel and substantially perpendicular up to about + or −10 degrees from true parallel and true perpendicular.

As used herein, the term “clinician” refers to a doctor, nurse, or other care provider and may include support personnel. In the following description, well-known functions or construction are not described in detail to avoid obscuring the present disclosure in unnecessary detail.

As will be described in detail below, provided is a robotic surgical assembly configured to be attached to a surgical robotic arm. The robotic surgical assembly includes an instrument drive unit having, for example, but not limited to, a motor configured to rotate an electromechanical instrument about a longitudinal axis thereof, and a carriage configured to slidably couple the instrument drive unit and the electromechanical instrument to the surgical robotic arm. The rotation of the electromechanical instrument may be selectively inhibited by a locking mechanism provided in the carriage. The locking mechanism may be purely mechanical and manually activated, electromechanical, or purely electrical.

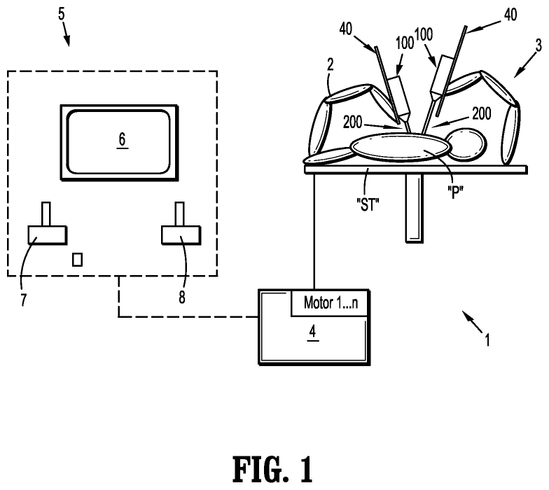

Referring initially to FIG. 1, a surgical system, such as, for example, a robotic surgical system 1, generally includes one or more surgical robotic arms 2, 3, a control device 4, and an operating console 5 coupled with control device 4. Any of the surgical robotic arms 2, 3 may have a robotic surgical assembly 100 coupled thereto. In some aspects, the robotic surgical assembly 100 may be removably attached to a slide rail 40 of one of the surgical robotic arms 2, 3. In certain aspects, the robotic surgical assembly 100 may be fixedly attached to the slide rail 40 of one of the surgical robotic arms 2, 3.

Operating console 5 includes a display device 6, which is set up to display three-dimensional images; and manual input devices 7, 8, by means of which a clinician (not shown), is able to telemanipulate the robotic arms 2, 3 in a first operating mode, as known in principle to a person skilled in the art. Each of the robotic arms 2, 3 may be composed of any number of members, which may be connected through joints. The robotic arms 2, 3 may be driven by electric drives (not shown) that are connected to control device 4. The control device 4 (e.g., a computer) is set up to activate the drives, for example, by means of a computer program, in such a way that the robotic arms 2, 3, the attached robotic surgical assembly 100, and thus an electromechanical surgical instrument 200 execute a desired movement according to a movement defined by means of the manual input devices 7, 8. The control device 4 may also be set up in such a way that it regulates the movement of the robotic arms 2, 3 and/or of the drives.

The robotic surgical system 1 is configured for use on a patient “P” positioned (e.g., lying) on a surgical table “ST” to be treated in a minimally invasive manner by means of a surgical instrument, e.g., the electromechanical surgical instrument 200. The robotic surgical system 1 may also include more than two robotic arms 2, 3, the additional robotic arms likewise connected to the control device 4 and telemanipulatable by means of the operating console 5. A surgical instrument, for example, the electromechanical surgical instrument 200, may also be attached to any additional robotic arm(s).

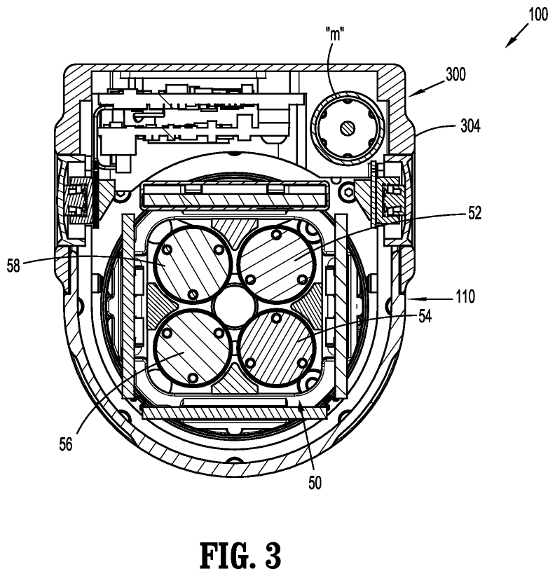

With reference to FIGS. 1-3, the control device 4 may control one or more motors, e.g., motors (Motor 1 . . . n), each motor configured to drive movement of the robotic arms 2, 3 in any number of directions. Further, the control device 4 may control an instrument drive unit 110 of the robotic surgical assembly 100 including motors 52, 54, 56 and 58 of a motor pack 50 (FIG. 3) disposed within a sterile barrier housing 130 of the instrument drive unit 110. The motors 52, 54, 56 and 58 of the motor pack 50 drive various operations of an end effector 202 of the electromechanical surgical instrument 200.

For a detailed discussion of the construction and operation of a robotic surgical system, reference may be made to U.S. Pat. No. 8,828,023, the entire contents of which are incorporated by reference herein.

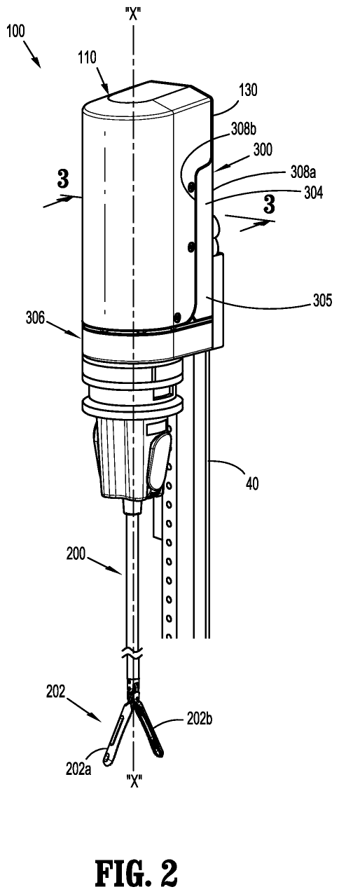

With reference to FIGS. 2 and 3, the robotic surgical assembly 100 includes the instrument drive unit 110, an instrument holder or carriage 300 for slidably coupling the instrument drive unit 110 to the rail 40, and an electromechanical surgical instrument such as electromechanical surgical instrument 200 configured to be drivingly coupled to the instrument drive unit 110. The instrument drive unit 110 transfers power and actuation forces from its motors 52, 54, 56, 58 to driven members (not shown) of the electromechanical surgical instrument 200 to ultimately drive movement of components of the end effector 202 of the electromechanical surgical instrument 200, for example, a movement of a knife blade (not shown) and/or a closing and opening of jaw members 202a, 202b of the end effector 202, the actuation or firing of a stapler, and/or the activation or firing of an electrosurgical energy-based instrument, or the like. The motor assembly or motor pack 50 of the instrument drive unit 110 is rotated by a motor “M” supported in the carriage 300 and transfers its rotational motion to the electromechanical surgical instrument 200.

The carriage 300 of the surgical assembly 100 functions to actuate a rotation of the motor assembly 50 of the instrument drive unit 110 and includes a back member 304 and an outer member or housing 306 extending laterally (e.g., perpendicularly) from an end 305 of the back member 304. In some aspects, the housing 306 may extend at various angles relative to the back member 304 and from various portions of the back member 304. The back member 304 has a first side 308a and a second side 308b, opposite to the first side 308a. The first side 308a of the back member 304 is detachably connectable to the rail 40 of the robotic arm 2 to enable the carriage 300 to slide or translate along the rail 40 of the robotic arm 2. The second side 308b of the back member 304 is configured to non-rotatably support the housing or outer shell 130 of the instrument drive unit 110.

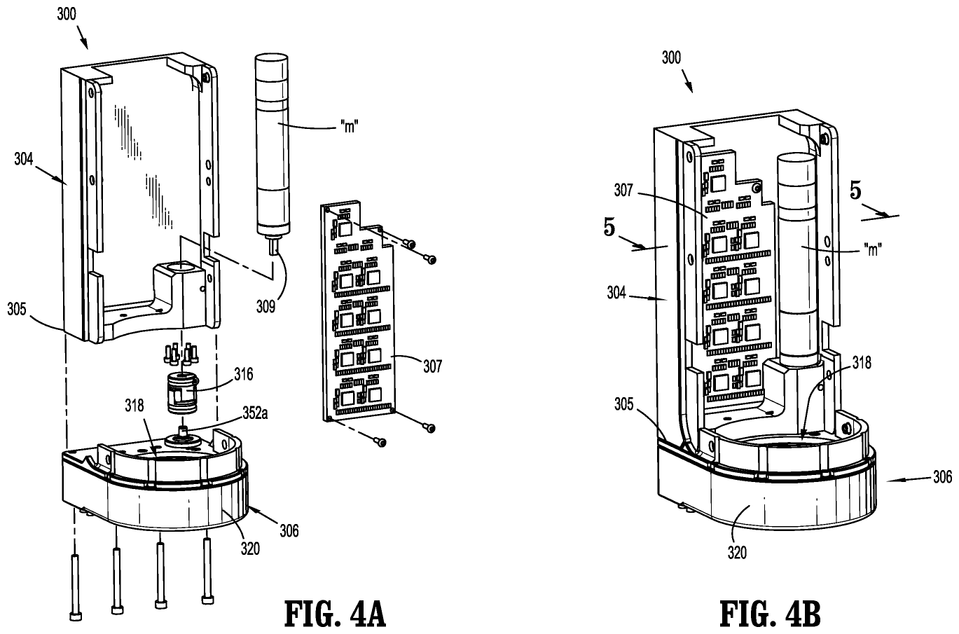

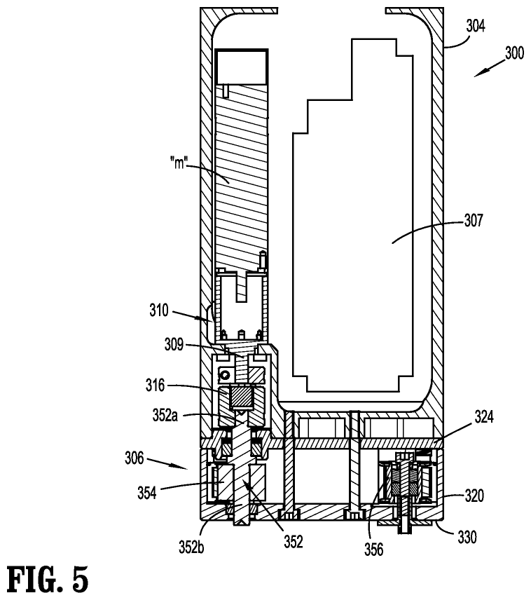

With reference to FIGS. 3-6, the carriage 300 supports or houses a motor, such as, for example, the canister motor “M” therein. The motor “M” receives controls and power from the control device 4 (FIG. 1) to ultimately rotate the motor assembly 50 (FIG. 3) of the instrument drive unit 110 and the attached surgical instrument 200, as will be described in detail below. In some aspects, the carriage 300 may include a printed circuit board 307 in electrical communication with the motor “M” to control an operation of the motor “M” of the carriage 300. The carriage 300 has a rotatable drive shaft 309 extending from the motor “M” and longitudinally through the carriage 300.

The housing 306 of the carriage 300 defines a channel 318 therethrough configured to rotatably receive and support the instrument drive unit 110 therein. The housing 306 has a generally oblong semicircular shape, but in some aspects, the housing 306 may assume a variety of shapes, such as, for example, C-shaped, U-shaped, V-shaped, hook-shaped, or the like. The housing 306 of the carriage 300 is further configured to house or retain the components of a drive assembly 310 of the carriage 300. The housing 306 generally includes a sidewall 320 defining an enclosure therein, and a top plate 324 connected to a top portion of the sidewall 320.

The drive assembly 310 of the carriage 300 is configured to transfer a rotation of the drive shaft 309 of the motor “M” into rotational motion of the motor assembly 50 of the instrument drive unit 110 when the instrument drive unit 110 is operably received within the carriage 300. The drive assembly 310 includes a driven shaft 352 rotatably disposed within the housing 306. The driven shaft 352 has a proximal end 352a extending proximally through the top plate 324 of the housing 306, and a distal end 352b extending distally through a base 330 of the housing 306. The proximal end 352a of the driven shaft 352 is non-rotatably connected to a shaft coupling 316 of the carriage 1104 such that rotation of the drive shaft 309 of the motor “M” causes the shaft coupling 316 to rotate and, in turn, the driven shaft 352 of the drive assembly 310 to rotate.

The drive assembly 310 includes a first pulley 354 and a second pulley 356; each disposed within the housing 306, and specifically at respective opposing corners thereof. The distal end 352b of the driven shaft 352 is non-rotatably connected to the first pulley 354 such that rotation of the driven shaft 352 effects rotation of the first pulley 354 relative to the housing 306. The first and second pulleys 354, 356 may be selectively movable within the housing 306 to different locations of the housing 306. The first and second pulleys 354, 356 may each be in the form of gears, such as, for example, spur gears having teeth 358 extending radially from a periphery thereof. In some aspects, the first and second pulleys 354, 356 may have smooth outer surfaces without teeth.

The drive assembly 310 further includes a drive strap or belt 360 rotatably and/or translatably received within the housing 306. The belt 360 is a closed loop and fabricated from a pliable material such that the belt 360 may be manipulated into any suitable shape. In particular, the belt 360 takes on the oblong semicircular shape of the housing 306 upon being received in the housing 306. In some aspects, the belt 360 may be formed from a rigid material and have a permanent oblong semicircular shape corresponding to the shape of the housing 306. The belt 360 may have teeth 362 extending from an inner surface thereof. The belt 360 is wrapped around the first and second pulleys 354, 356 such that the teeth 362 of the belt 360 are in operable engagement with the teeth 358 of the first and second pulleys 354, 356. In this way, rotation of the first pulley 354, caused by actuation of the motor “M” of the carriage 300, causes the belt 360 to rotate around the first and second pulleys 354, 356. The second pulley 356 acts as an idler pulley to guide the belt 360 around the inner periphery of the sidewall 320 of the housing 306. It is contemplated that the second pulley 356 may be selectively moved to a plurality of positions to effect the tension on/off the belt 360.

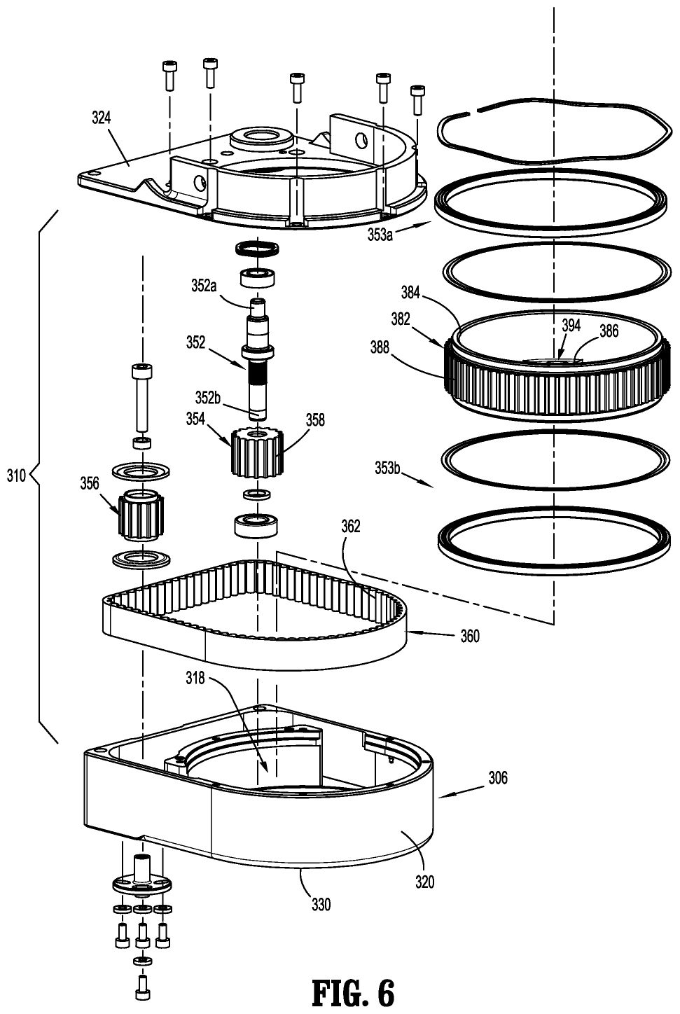

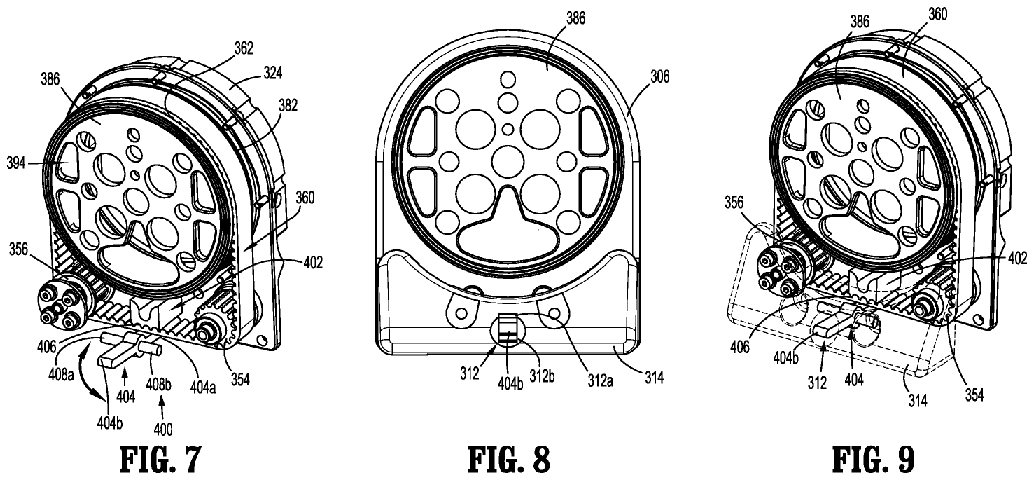

With reference to FIG. 6, the drive assembly 310 includes a cup-shaped annular member 382 rotatably disposed within the channel 318 of the housing 306 between the first and second bearings 353a, 353b of the drive assembly 310. The annular member 382 includes a ring 384 and an annular base plate or disc 386 (see also FIGS. 7-9) disposed within the ring 384. The ring 384 has a plurality of teeth 388 extending radially from an outer surface thereof. With the annular member 382 rotatably seated between the first and second bearings 353a, 353b of the drive assembly 310, the teeth 388 of the annular member 382 are in operable engagement with the teeth 362 of the belt 360. In this regard, movement of the belt 360 along the inner periphery of the sidewall 320 of the housing 306 by rotation of the first pulley 354 causes the annular member 382 to rotate within the channel 318 of the housing 306. The annular base plate 386 defines a plurality of holes 394 therethrough configured for receipt of various drive shafts (not shown) of the instrument drive unit 110. With the drive shafts of the instrument drive unit 110 extending through the holes 394 of the annular base plate 386, rotation of the annular member 382 via belt 360 results in rotation of the motor assembly 50 (FIG. 3) of the instrument drive unit 110 relative to the housing 306 of the carriage 300.

With reference to FIGS. 7-9, the carriage 300 may further include a mechanical locking assembly 400 for selectively locking the belt 360, and therefore selectively inhibiting rotation of the motor pack 50 of the instrument drive unit 110 during emergency situations and/or during a power outage. The mechanical locking assembly 400 is supported in the housing 306 of the carriage 300 and includes a lock pad 402 and a lever arm 404. The lock pad 402 is disposed interiorly of the belt 360 and is fixed in spaced relation to the teeth 362 of the belt 360. The lock pad 402 has a planer face defining a plurality of teeth 406 configured to selectively meshingly engage the teeth 362 of the belt 360.

The lever arm 404 is pivotably supported in the housing 306 via a pair of lateral pivot pins 408a, 408b extending from opposite sides of the lever arm 404. The lever arm 404 has a first end 404a disposed outside of the belt 460 and in diametric opposition from the planar face 406 of the lock pad 402 such that a section of the belt 460 is disposed between the lock pad 402 and the first end 404a of the lever arm 404. A second end 404b of the lever arm 404 extends through a slot 312 defined through a base portion 314 of the housing 306. The slot 312 has a first end 312a sized to fix the second end 404b of the lever arm 404 therein when the lever arm 404 is in a first, non-locking state, and a second end 312b sized to fix the second end 404b of the lever arm 404 therein when the lever arm 404 is in a second, locking state.

In use, to prevent the motor pack 50 (FIG. 3) of the instrument drive unit 110, and accordingly the attached surgical instrument 200, from rotating about the longitudinal axis “X” (FIG. 2), a clinician may grasp the second end 404b of the lever arm 404 of the mechanical locking assembly 400 and apply a threshold force sufficient to dislodge the second end 404b of the lever arm 404 from the first end 312a of the slot 312. The clinician pivots the second end 404b of the pivot arm 404 through the slot 312 and into locking engagement with the second end 312b of the slot 312 to fix the pivot arm 404 in the second, locking state, as shown in FIGS. 7-9. As the lever arm 404 is pivoted, about a pivot axis defined through the pivot pins 408a, 408b, the first end 404a of the lever arm 404 engages the outer surface of the belt 460 to deflect the belt 460 out of its normal state to press the teeth 362 of the belt 360 into meshing engagement with the teeth 406 of the lock pad 402. With the teeth 362 of the belt 360 maintained in meshing engagement with the teeth 406 of the lock pad 402, the belt 360 is held stationary, thereby fixing the rotational orientation of the motor pack 50 of the instrument drive unit 110 and the attached surgical instrument 200. To unlock the belt 360 from the mechanical locking mechanism 400, the clinician pivots the lever arm 404 from the second state back to the first state, whereby the first end 404a of the lever arm 404 disengages the belt 360, allowing the belt 360 to flex outwardly to assume its normal state, in which the teeth 362 of the belt 360 are spaced from the teeth 406 of the lock pad 402.

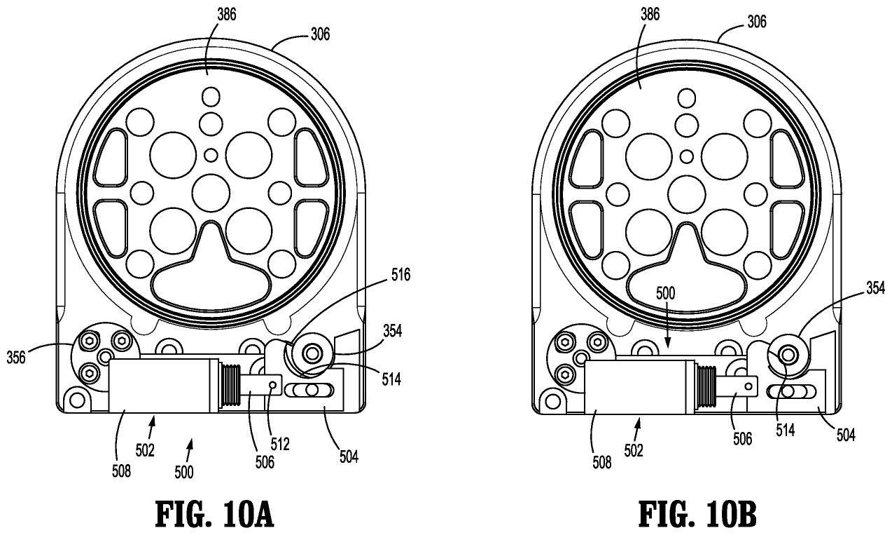

With reference to FIGS. 10A and 10B, the carriage 300 of the robotic surgical assembly 100 may include an alternative locking mechanism to the mechanical locking mechanism 400 of FIGS. 7-9. In particular, the carriage 300 may have an electromechanical locking mechanism 500 that includes a solenoid 502 and a pawl 504 configured to selectively engage the pulley 354 of the drive assembly 310. In aspects, the pawl 504 may be configured to selectively engage the pulley 356. The solenoid 502 may be in electrical communication with a source of current, such as, for example, control device 4 (FIG. 1), which under normal operating conditions supplies a current to the solenoid 502. The solenoid 502 may be a linear solenoid having a plunger 506 that extends from a housing 508 of the solenoid 502 a select distance when the solenoid 502 is receiving a threshold current. Upon a ceasing of the current, the solenoid 502 is configured to extend the plunger 506 from the housing 508 to actuate locking mechanism 500. In other aspects, the plunger 506 may be configured to retract from the housing 508 to actuate the locking mechanism 500 upon the ceasing of current to the solenoid 502. In aspects, the solenoid 502 may be any other suitable type of solenoid, such as a rotary solenoid. In aspects, the solenoid 502 may be equipped with a manual override, such as a handle (not explicitly shown) that extends from the plunger 506 and out of the carriage 300 for manual actuation thereof.

The pawl 504 is supported in the housing 306 of the carriage 300 and is permitted to slide along an axis that is coaxial with the plunger 506 of the solenoid 502. The pawl 504 is fixed to an end 512 of the plunger 506 of the solenoid 502 and is configured to slide with or otherwise in response to a sliding of the plunger 506. The pawl 504 has an arcuate surface 514 at least partially surrounding the pulley 354. The arcuate surface 514 of the pawl 504 defines one or more teeth 516 configured to selectively engage the gear teeth of the pulley 354.

In use, under normal operating conditions, current is continuously supplied to the solenoid 502 of the electromechanical locking mechanism 500, whereby the solenoid 502 maintains the plunger 506 in a retracted position, as shown in FIG. 10A. When current is no longer flowing to the solenoid 502, such as in a power loss condition, the solenoid 502 is deactivated, which results in the plunger 506 of the solenoid 502 advancing further out of the housing 508 toward the pulley 354. The teeth 516 of the pawl 504 engage the teeth of the pulley 354, thereby fixing the rotational orientation of the pulley 354, and therefore the motor pack 50 (FIG. 3) of the instrument drive unit 110 and the attached surgical instrument 200. When power returns, the current may begin flowing back to the solenoid 502 to activate the solenoid 502, whereby the plunger 506 retracts from the extended position back to the retracted position, thereby disengaging the teeth 516 of the pawl 504 from the pulley 354. With the pawl 504 disengaged from the pulley 354, the pulley 354 is then free to rotate along with the power pack 50 of the instrument drive unit 110 and the attached surgical instrument 200.

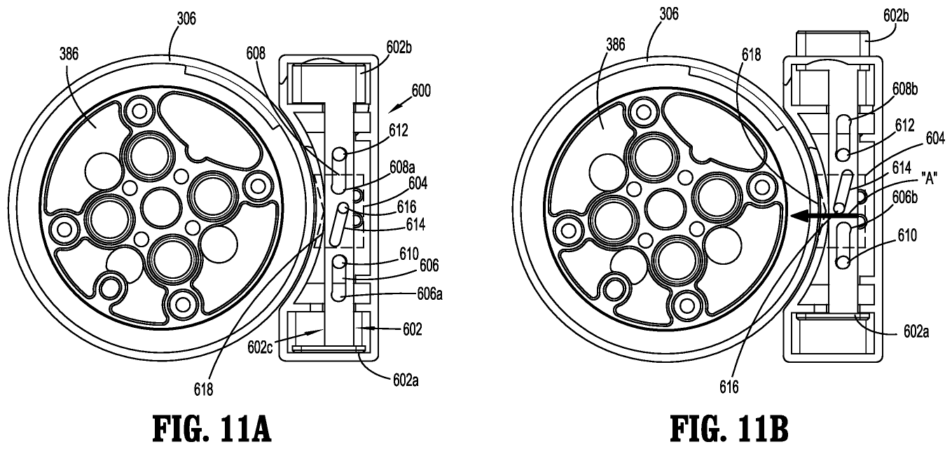

With reference to FIGS. 11A and 11B, the carriage 300 of the robotic surgical assembly 100 may include an alternative locking mechanism to the locking mechanisms 400, 500 of FIGS. 7-10B. In particular, the carriage 300 may have a mechanical braking assembly 600 configured to selectively engage the annular member 382 either directly, or indirectly via the belt 360, and includes a slider 602 and a brake 604 operably coupled to the slider 602. The slider 602 is slidably supported in the housing 306 and is configured to slide relative to the housing 306 along a longitudinal axis defined by the slider 602 between a first or non-locking position (FIG. 11A) and a second or locking position (FIG. 11B).

The slider 602 has a first end 602a defining a first manually-activated button, a second end 602b defining a second manually-activated button, and an elongated shaft 602c extending therebetween. The second button 602b may have a discrete color from the remainder of the slider 602 and/or include a light (e.g., an LED) indicating that the brake 604 is in a deployed or non-deployed state. In aspects, a sensor, such as a hall effect sensor, or a switch, may be provided to detect whether the brake 604 is deployed.

The elongate shaft 602c defines first and second elongate slots 606, 608 through which respective pins 610, 612 of the housing 306 extend. Each of the elongate slots 606, 608 has a first end 606a, 608a defining a first detent, and a second end 606b, 608b defining a second detent each configured to selectively capture the respective pin 610, 612 therein when the slider 602 is in one of the first or second positions. The elongate shaft 602c of the slider 602 further includes a cam slot 614 disposed between the first and second elongate slots 606, 608 and being angled (e.g., non-parallel) relative to the longitudinal axis of the slider 602. Received in the cam slot 614 is a pin or projection 616 extending upwardly from the brake 604.

The brake 604 is disposed under the elongate shaft 602c of the slider 602 and is configured to slide in a direction perpendicular to the longitudinal axis of the slider 602 in response to a translation of the slider 602. The brake 604 may be a block of rubber or any other suitable material and has an arcuate face 618 that faces and is spaced from the outer surface of the annular member 382 when the slider 602 is in the first position.

In use, to fix the rotational orientation of the annular member 382, and thus the motor pack 50 of the instrument drive unit 110, a clinician may exert a threshold force on the first end 602a of the slider 602 to dislodge the pins 610, 612 from the detents 606b, 608b of the elongate slots 606, 608 of the slider 602, whereby the slider 602 translates from the first position (FIG. 11A) toward the second position (FIG. 11B), in which the pins 610, 612 lockingly engage the detents 606a, 608a of the elongate slots 606, 608 of the slider 602. In aspects, the slider 602 may be moved via an electromechanical device, such as a solenoid, rather than being manually-activated. As the slider 602 translates toward the second position, the cam slot 614 of the slider 602 cams the projection 616 of the brake 604 to slide the brake 604 in the direction indicated by arrow “A” in FIG. 11B to engage the arcuate face 618 of the brake 604 with the outer surface of the annular member 382. The frictional engagement between the arcuate face 618 of the brake 604 and the outer surface of the annular member 382 resists rotation of the annular member 382, and therefore the motor pack 50 of the instrument drive unit 110 and the attached surgical instrument 200.

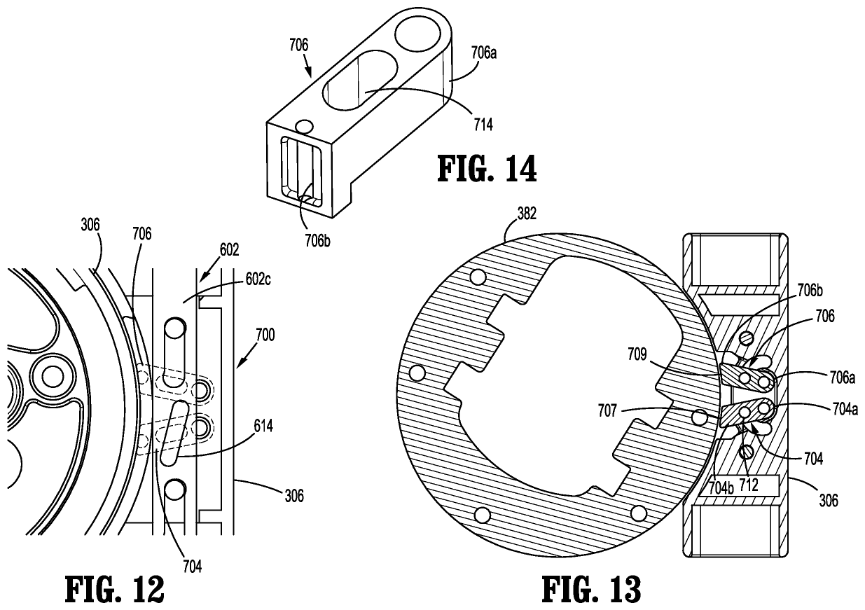

With reference to FIGS. 12-14, the carriage 300 of the robotic surgical assembly 100 may include an alternative, but similar locking mechanism 700 to the locking mechanism 600 of FIGS. 11A and 11B. In particular, the locking mechanism 700 is self-locking and may include the slider 602, a pair of pivotable brakes 704, 706, and a cam member (not explicitly shown) operably coupling the slider 602 to the pair of pivotable brakes 704, 706. The cam slot 614 in the elongate shaft 602c of the slider 602 receives a pin or projection (not explicitly shown) extending upwardly from the cam member, such that axial movement of the slider 602 drives a translation of the cam member in a direction perpendicular to the longitudinal axis of the slider 602.

The pair of pivotable brakes 704, 706 are positioned adjacent one another at an acute angle. Each of the pivotable brakes 704, 706 has a first end 704a, 706a pivotably coupled to the housing 306, and a second end 704b, 706b defining an angled face 707, 709 configured to selectively frictionally-engage the outer surface of the annular member 382. The cam member has a pair of surface features (not shown) received in cam slots 712, 714 defined in each of the respective first and second pivotable brakes 704, 706. The surface features of the cam member are configured to slide through the cam slots 712, 714 of the pivotable brakes 704, 706 during an actuation of the slider 602 to pivot the second ends 704b, 706b of the pivotable brakes 704, 706 into engagement with the outer surface of the annular member 382.

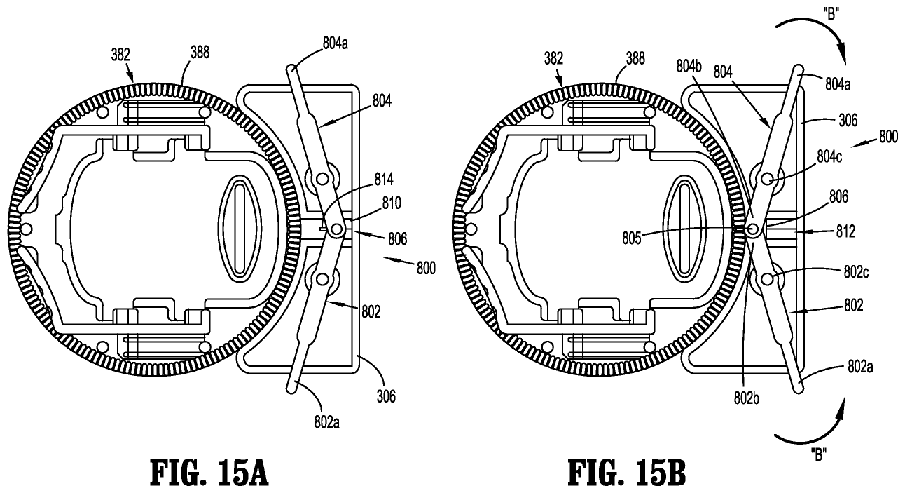

With reference to FIGS. 15A and 15B, the carriage 300 may include an alternative locking mechanism to the locking mechanisms 400, 500, 600, 700 of FIGS. 7-14. In particular, the carriage 300 may have a mechanical locking mechanism 800 configured to selectively engage the annular member 382 either directly, or indirectly via the belt 360, and includes a pair of levers or toggle arms 802, 804 and a pawl 806 operably engaged to the toggle arms 802, 804. Each of the toggle arms 802, 804 has an elongated shape and has a first end 802a, 804a exposed from the housing 306 to enable manual actuation by a clinician, and a second end 802b, 804b. The second ends 802b, 804b of the toggle arms 802, 804 are pivotably coupled to one another at a common pivot point 805 that extends through the pawl 806. The toggle arms 802, 804 are each pinned to the housing 306 at an intermediate portion 802c, 804c thereof. The mechanical locking mechanism 800 may be activated upon actuation of toggle arm 802, toggle arm 804, or both toggle arms 802, 804 simultaneously.

The pawl 806 of the mechanical locking mechanism 800 has a body 810 slidably received in a longitudinal track 812 defined in the housing 306, and a surface feature 814, such as, for example, a tooth, spike, fin, or the like, extending from the body 810. The second ends 802b, 804b of the toggle arms 802, 804 are each pivotably coupled to the body 810 of the pawl 806. The surface feature 814 of the pawl 806 is sized to form an interference fit between adjacent teeth 388 of the annular member 382.

In use, to fix the rotational orientation of the annular member 382, and thus the motor pack 50 of the instrument drive unit 110, a clinician may exert a threshold force on the first end 802a, 804a of each of the toggle arms 802, 804 to rotate each of the toggle arms 802, 804 about the pinned connection, in the direction indicated by arrow “B” in FIG. 15B. In aspects, the toggle arms 802, 804 may be moved via an electromechanical device, such as a solenoid, rather than being manually-activated. The second end 802b, 804b of each of the toggle arms 802, 804 drags the pawl 806 along the track 812 to a locking position, as shown in FIG. 15B, in which the surface feature 814 of the pawl 806 enters a space defined between adjacent teeth 388 of the annular member 382. The interference fit between the pawl 806 and the teeth 388 of the annular member 382 resists rotation of the annular member 382, and therefore the motor pack 50 of the instrument drive unit 110 and the attached surgical instrument 200.

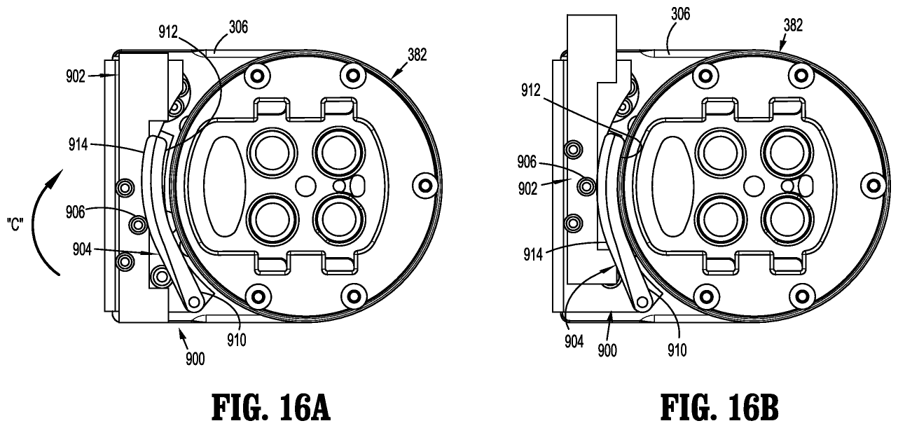

With reference to FIGS. 16A and 16B, the carriage 300 of the robotic surgical assembly 100 may include an alternative locking mechanism 900 to the locking mechanisms 400, 500, 600, 700, 800 of FIGS. 7-15B. In particular, the carriage 300 may have a mechanical braking mechanism 900 configured to selectively engage the annular member 382 either directly, or indirectly via the belt 360, and includes a cam slider 902 and a brake 904 operably coupled to the cam slider 902. The cam slider 902 is slidably supported in the housing 306 and is configured to slide relative to the housing 306 along a longitudinal axis defined by the cam slider 902 between a first or non-locking position (FIG. 16A) and a second or locking position (FIG. 16B). The cam slider 902 has a cam feature 906, such as, for example, a ball bearing, a roller, a ramped surface, or the like, that is configured to slide or roll along a surface 914 of the brake 904 to cam the brake 904.

The brake 904 may be an elongated rocker arm having an end 910 pivotably coupled to the housing 306. The brake 904 has a concave front face 912 partially encircling the outer surface of the annular member 382, and a convex rear face 914 engaged with the camming feature 906 of the slider 902. In aspects, the brake 904 may be a friction brake, a spring loaded clutch or a mechanical engagement locking brake.

In use, to fix the rotational orientation of the annular member 382, and thus the motor pack 50 of the instrument drive unit 110, a clinician may manually translate the slider 902 from the first position (FIG. 16A) toward the second position (FIG. 16B). In aspects, the slider 902 may be moved via an electromechanical device, such as a solenoid, rather than being manually-activated. As the slider 902 translates toward the second position, the camming feature 906 of the slider 902 slides along the convex rear face 914 of the brake 904 to pivot the brake 904 in the direction indicated by arrow “C” in FIG. 16A to engage the concave front face 912 of the brake 904 with the outer surface of the annular member 382. The frictional engagement between the concave front face 912 of the brake 904 and the outer surface of the annular member 382 resists rotation of the annular member 382, and therefore the motor pack 50 of the instrument drive unit 110 and the attached surgical instrument 200.

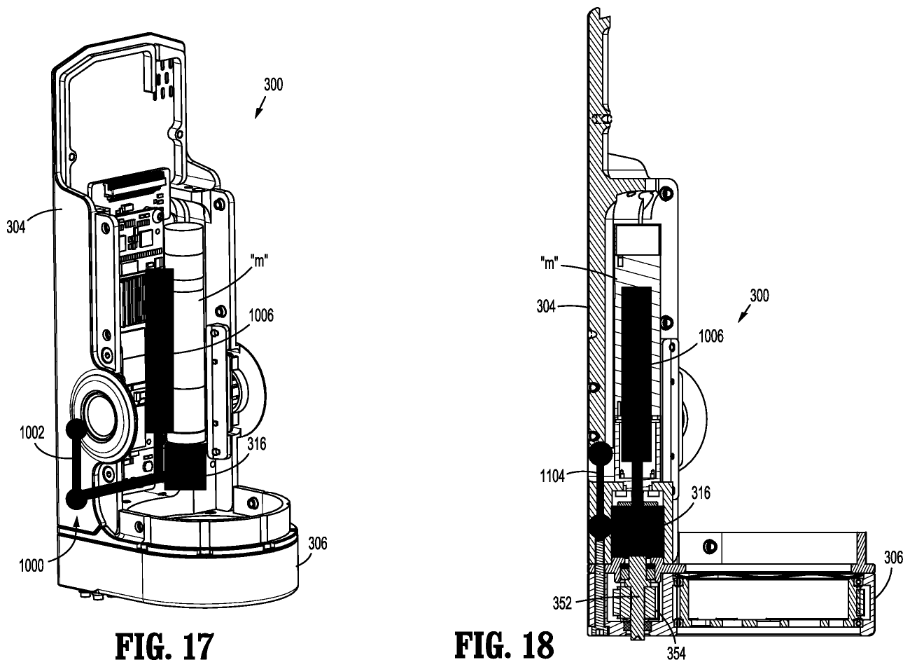

With reference to FIGS. 17 and 18, another mechanism 1000 for selectively locking the rotational orientation of the motor pack 50 (FIG. 3) of the instrument drive unit 110 is provided. In particular, the locking mechanism 1000 includes a manual slider 1002 disposed on a lateral side of the back member 304 of the carriage 300 and operably coupled to a brake (not explicitly shown). The brake may be a friction brake, a spring-loaded clutch, or a mechanically-engaged locking brake disposed along a motor drive axis and configured to selectively engage the shaft coupling/motor drive coupling 316. To activate the brake, the slider 1002 is moved from a first or proximal position to a second or distal position, in which the brake engages the motor drive coupler 316, thereby preventing rotation thereof, and therefore also preventing rotation of the motor pack 50 of the instrument drive unit 110 and the attached surgical instrument 200. A secondary solenoid 1006 and/or a motor/servo drive may be provided in association with the brake as an alternative means of activating the brake.

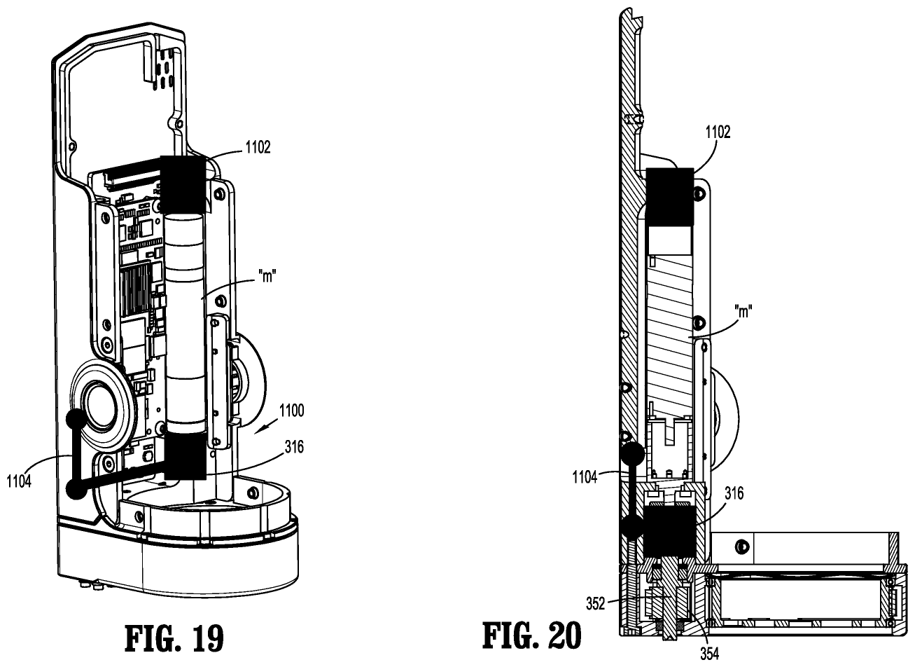

With reference to FIGS. 19 and 20, another mechanism 1100 for selectively locking the rotational orientation of the motor pack 50 (FIG. 3) of the instrument drive unit 110 is provided, similar to the locking mechanism 1000 of FIGS. 17 and 18. A brake 1102 may be integrated into the motor “M” for selectively holding the motor “M” during a power off condition. A manual slider 1104 may be provided in operable engagement with the motor drive coupler 316. The manual slider 1104 is configured to move between a first or distal position, in which the motor drive coupler 316 is operably engaged with the motor “M,” and a second or proximal position, in which the slider 1104 disengages the motor drive coupler 116 from the motor “M” to allow a clinician to manually rotate the surgical instrument 200 during a power off condition if needed despite the motor “M” being locked out by the integrated brake 1102.

Any of the disclosed mechanical and electro-mechanical drive assemblies may also include the use of a lever, a rotating knob, a fulcrum or an actuator that applies an additional mechanical advantage to the end user to initiate breaking. This may be accomplished through an integrated lever, actuator, or mechanism, or an external tool to provide a similar function.

Any of the disclosed mechanical and electro-mechanical drive assemblies may also include the use of a frictional or magnetic slip clutch mechanism to provide a torsion threshold limit that can be overcome.

Any of the disclosed mechanical and electro-mechanical drive assemblies may also include an external locking pin, tool, c-clip, flat bar or profiled bar in the stationary portion or the drive that can be engaged or mated into a key, slot, flat, tooth, undercut or profile on the rotating portion of the drive to provide a less complex and costly braking with a reduced package size for the drive.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A robotic surgical assembly, comprising:
an instrument drive unit configured to drive operations of a surgical instrument; and
a carriage configured to be slidably coupled to a robotic arm and to support the instrument drive unit, the carriage including:
a drive assembly having:
a motor;
a pulley operably coupled to the motor; and
a drive belt coupled to the pulley and configured to be operably coupled to the surgical instrument, such that the surgical instrument rotates in response to a rotation of the pulley; and
a locking assembly configured to selectively lock the drive assembly to prevent rotation of the surgical instrument.

2. The robotic surgical assembly according to claim 1, wherein the locking assembly includes:
a lock pad disposed adjacent a first side of the drive belt; and
a lever arm having a first end disposed on a second side of the drive belt, wherein the lever arm is configured to pivot between a first position, in which the drive belt is in spaced relation to the lock pad, and a second position, in which the first end of the lever arm presses the drive belt into engagement with the lock pad to resist travel of the drive belt.

3. The robotic surgical assembly according to claim 2, wherein the first side of the drive belt has a plurality of teeth, and the lock pad has a tooth configured for receipt in adjacent teeth of the plurality of teeth of the drive belt when the lever arm is in the second position.

4. The robotic surgical assembly according to claim 2, wherein the carriage includes a housing defining a slot, the slot having a first end configured for detachable receipt of a second end of the lever arm when the lever arm is in the first position, and a second end configured for detachable receipt of the second end of the lever arm when the lever arm is in the second position.

5. The robotic surgical assembly according to claim 1, wherein the instrument drive unit includes:
a housing configured to be rotationally fixed relative to the carriage; and
a motor pack rotatably supported in the housing.

6. The robotic surgical assembly according to claim 5, wherein the motor pack is configured to non-rotatably couple to the surgical instrument.

7. The robotic surgical assembly according to claim 6, wherein the drive belt is configured to be non-rotatably coupled to the motor pack, such that the motor pack and the surgical instrument rotate about a longitudinal axis defined by the surgical instrument upon rotation of the pulley.

8. The robotic surgical assembly according to claim 1, wherein the locking assembly includes:
a solenoid; and
a pawl associated with the solenoid and disposed adjacent the pulley, wherein the solenoid is configured to move the pawl between a first position, in which the pawl is disengaged from the pulley, and a second position, in which the pawl is engaged to the pulley to resist rotation of the pulley.

9. The robotic surgical assembly according to claim 8, wherein the solenoid includes:
a housing; and
a plunger coupled to the housing and having an end fixed to the pawl, such that the pawl moves between the first and second positions in response to movement of the plunger relative to the housing.

10. The robotic surgical assembly according to claim 9, wherein the pawl has an arcuate surface defining a tooth configured to engage the pulley when the pawl is in the second position.

11. A robotic surgical system, comprising:
a surgical instrument defining a longitudinal axis;
an instrument drive unit configured to transmit rotational forces to the surgical instrument while the surgical instrument is coupled to the instrument drive unit, the instrument drive unit having a motor pack configured to non-rotatably couple to the surgical instrument; and
a carriage supporting the instrument drive unit and the surgical instrument while the surgical instrument is coupled to the instrument drive unit, the carriage including:
a back member;
a housing extending from the back member;
a drive assembly having:
a motor supported in the back member;
a pulley supported in the housing and operably coupled to the motor; and
a drive belt supported in the housing and coupled to the pulley, the drive belt being configured to non-rotatably couple to the motor pack, such that the surgical instrument rotates in response to a rotation of the pulley; and
a locking assembly configured to selectively lock the drive assembly to prevent rotation of the surgical instrument.

12. The robotic surgical system according to claim 11, wherein the locking assembly includes:
a lock pad disposed adjacent a first side of the drive belt; and
a lever arm having a first end disposed on a second side of the drive belt, wherein the lever arm is configured to pivot between a first position, in which the drive belt is in spaced relation to the lock pad, and a second position, in which the first end of the lever arm presses the drive belt into engagement with the lock pad to resist travel of the drive belt.

13. The robotic surgical system according to claim 12, wherein the first side of the drive belt has a plurality of teeth, and the lock pad has a tooth configured for receipt in adjacent teeth of the plurality of teeth of the drive belt when the lever arm is in the second position.

14. The robotic surgical system according to claim 12, wherein the housing of the carriage defines a slot having a first end configured for detachable receipt of a second end of the lever arm when the lever arm is in the first position, and a second end configured for detachable receipt of the second end of the lever arm when the lever arm is in the second position.

15. The robotic surgical system according to claim 11, further comprising a rail configured to slidably support the back member of the carriage thereon.

16. The robotic surgical system according to claim 11, wherein the locking assembly includes:
a solenoid; and
a pawl associated with the solenoid and disposed adjacent the pulley, wherein the solenoid is configured to move the pawl between a first position, in which the pawl is disengaged from the pulley, and a second position, in which the pawl is engaged to the pulley to resist rotation of the pulley.

17. The robotic surgical system according to claim 16, wherein the solenoid includes:
a housing; and
a plunger coupled to the housing of the solenoid and having an end fixed to the pawl, such that the pawl moves between the first and second positions in response to movement of the plunger relative to the housing of the solenoid.

18. The robotic surgical system according to claim 16, wherein the pawl has an arcuate surface defining a tooth configured to engage the pulley when the pawl is in the second position.

19. The robotic surgical system according to claim 11, wherein the locking assembly includes:
a slider slidably supported in the housing and defining a cam slot; and
a brake having a projection received in the cam slot, the slider configured to move the brake between a first position, in which the motor pack is free to rotate, and a second position, in which the brake resists rotation of the motor pack.

20. The robotic surgical system according to claim 19, wherein the brake moves between the first and second positions along an axis that is perpendicular to a longitudinal axis defined by the slider.
